(19) United States
(12) Patent Application Publication
(10) Pub. No.: US 2023/0389632 A1
(43) Pub. Date: Dec. 7, 2023

(54) Ultrasound Probe Hand Shield

(71) Applicant: Wisconsin Alumni Research Foundation, Madison, WI (US)

(72) Inventors: Alejandro Roldán-Alzate, Middleton, WI (US); Sylvana García-Rodríguez, Middleton, WI (US); Mark Kliewer, Madison, WI (US); Timothy Walker, Madison, WI (US)

(21) Appl. No.: 17/833,178

(22) Filed: Jun. 6, 2022

(51) Int. Cl. A41D 13/08; A61B 8/00

(52) U.S. Cl. CPC A41D 13/085; A61B 8/4455; A61B 8/4422

(57) ABSTRACT

A hand shield may be installed onto an ultrasound probe to protect the operator from needle sticks. The transparent shield may be a disposable, sterilized film attachable to a frame which, when securely attached to the frame, blocks the needle from accidentally contacting and sticking the operator's hand during needle insertion. The shape and size of the frame avoids interference with the operator's hand and maintains normal operation of the ultrasound probe, thus recognizing the need for the angle of insonation (i.e., angle of the ultrasound beam relative to the tissue or organ of interest) to be adjusted and the ultrasound probe to be pressed into the tissue.

### STATEMENT REGARDING FEDERALLY SPONSORED RESEARCH OR DEVELOPMENT

—

### CROSS REFERENCE TO RELATED APPLICATION

—

### Background of the Invention

The present invention relates to personal protective equipment to protect the wearer from injury during medical procedures and in particular to a protective hand shield worn during ultrasound probe operation to protect the wearer's hand during simultaneous needle insertion.

An ultrasound transducer or ultrasound probe is a medical device allowing a healthcare professional to create an image or sonogram of organs, tissues, and other structures beneath the tissue and inside the body. A simple ultrasound transducer provides for a wand-like instrument that is passed over the surface of the body, or sometimes inserted into an orifice of the body, that emits sound waves and picks up the echoes that bounce off the organs, tissues, and structures of interest to create the image or sonogram.

The ultrasound transducer typically includes a transducer bar that holds an arrangement of piezoelectric crystals located near the skin-contacting face of the transducer that generates the ultrasound waves in response to an electric current applied to the piezoelectric crystals. The piezoelectric crystals also receive the echoes and sends them back to a computer as an electric current to produce the sonogram. The transducer bar is supported by an acoustically insulated housing to prevent other external echoes from affecting the sonogram. The housing of the transducer provides an operator handgrip so that the operator can effectively grip the transducer during movement of the transducer bar over the surface of the skin and over the target organ or tissue of interest.

There are three common types or shapes of ultrasound transducers used for imaging: linear, convex/curvilinear, and sector (phased) array. A linear ultrasound transducer has a linear piezoelectric crystal arrangement and produces a rectangular beam shape. A convex/curvilinear ultrasound transducer has a curvilinear piezoelectric crystal arrangement and produces a convex shaped beam. A sector (phased) array transducer has a linear or curvilinear piezoelectric crystal arrangement and produces a beam with a narrow point that expands to a triangular shape. The type of transducer chosen depends on the desired depth of the target organ or tissue of interest and application (e.g., visualization of tissue and organs such as the thyroid, breast, tendon, abdomen, heart, brain, nerve, musculoskeletal system; vascular examination; venipuncture; intraoperative laparoscopy; photoacoustic flow velocity imaging; body fat measurement; locomotive syndrome check; etc.).

Examples of these ultrasound transducers are manufactured by GE Healthcare and are found at <https://www.gehealthcare.com/products/ultrasound/ultrasound-transducers>, hereby incorporated by reference.

Ultrasound transducer operators (e.g., sonographers or physicians) are trained to use proper hand mechanics when operating the transducer to prevent fatigue, strain, and overuse injuries. For example, the operator must use the proper hand grip and the grip cannot be too tight. The transducer is often held at the base of the transducer to facilitate control and the operator may maintain contact with the patient's skin while scanning to maintain stability and prevent the transducer from moving or rotating out of the desired image location. For example, in one gripping orientation, the operator may contact the patient with the pinky and ring fingers and ulnar aspect of the hand while gripping the transducer with the thumb, index, and middle fingers providing stability and optimizing control.

During the ultrasound, the computer will provide a real time image which helps guide that operator to producing the optimal image. In some cases, the ultrasound is used during an interventional procedure (e.g., injection or aspiration of joints, tendons, or tendon shrouds; aspiration of cysts, fluid collections, and abscesses; peripheral nerve blocks/perineural injections; lavage and aspiration of tendon calcifications; fine-needle aspiration and biopsy; and foreign body retrieval) to diagnose, ensure therapeutic efficacy or procedural success, and to reduce procedural risk. The ultrasound requires looking back and forth at the ultrasound screen showing the real time image and the patient/ultrasound probe. These back and forth head movements add complexity to the procedure. The recorded sonogram may be later interpreted by an imaging specialist (e.g., radiologist or physician) who will report on the results of the image and provide a diagnosis.

### SUMMARY OF THE INVENTION

Ultrasound is useful for guiding interventional procedures such as aspirations, ablations, and biopsies, by providing real-time imaging to clearly visualize the pathway for a needle. Ultrasound guided interventional procedures are desirable because they can use a small and portable ultrasound machine, has lower medical costs, and poses no radiation hazard compared to other image guidance techniques. However, the technique has a steep learning curve and involves training the operator in proper ultrasound mechanics and guidance.

The present inventors have recognized that ultrasound guided interventional procedures increase the risk of accidental needle sticks, for example, during medical training when the medical trainee is performing a needle insertion during ultrasound scanning by the same medical trainee or a separate ultrasound operator. Moreover, the stabilization of the ultrasound probe often requires the operator's hand (i.e., ulnar aspect) to be placed on the surface of the patient's skin, close to the needle insertion site, increasing the risk of needle sticks.

Ultrasound guided interventional procedures may involve one or more operators holding the ultrasound transducer scanning and holding the needle for insertion, respectively. Whether one, two or more operators are involved in the procedure, coordination of the ultrasound probe simultaneously with the needle insertion runs the risk of accidental needle sticks. The ultrasound screen is often positioned outside the line of sight of the ultrasound probe or needle insertion site, therefore increasing the risk of needle sticks during back and forth head movements of the operator.

Multiple needle reinsertions and redirections during the ultrasound guided interventional procedure increases the risk of needlestick injuries and increases the risk of exposure to the operator of bloodborne pathogen transmission and must be reported and managed immediately to prevent serious injury and consequences.

In one embodiment, the invention may provide a shroud comprising a frame and replaceable transparent shield that may be installed onto an ultrasound probe to protect the operator from needle sticks. The transparent shield may be a disposable, sterilized film attachable to the frame which, when securely attached to the frame, blocks the needle from accidentally contacting and sticking the operator's hand during needle insertion. The shape and size of the frame avoids interference with the operator's hand and maintains normal operation of the ultrasound probe, thus recognizing the need for the angle of insonation (i.e., angle of the ultrasound beam relative to the tissue or organ of interest) to be adjusted and the ultrasound probe to be pressed into the patient's skin to provide optimal imaging.

In different embodiments, the hand shield assembly may allow for an ultrasound needle guide, attached to the ultrasound probe, to be accommodated and used with the probe.

In different embodiments, the hand shield assembly permit the operator to grip the ultrasound probe and will accommodate operator grip preferences. Further, the frame and transparent shield may permit the operator's hand to contact the patient's skin to stabilize the ultrasound probe during scanning as would be done during normal operation of the probe.

In different embodiments, the hand shield assembly may accommodate different shapes and sizes of ultrasound transducers and different shapes and sizes of needle guides.

Specifically, then, in one embodiment, the invention provides an ultrasound transducer hand shield for protecting a hand of a healthcare professional gripping a transducer in a scanning position with a front end of the transducer adjacent to tissue of a patient supporting an array of piezoelectric elements, and a rear end of the transducer supporting a hand grip extending along an axis of the transducer perpendicular to the array of piezoelectric elements. The hand shield comprises a shroud removably supported by the transducer to extend along the axis of the transducer and configured to extend at least partially around the transducer to create a physical barrier between the hand grip and a needle insertable into the tissue of the patient below the transducer; and a connector supporting a connection of the shroud at a front end of the transducer wherein the shroud extends around but does not contact the rear end of the transducer.

It is thus a feature of at least one embodiment of the invention to provide the protective benefits of personal protective equipment during ultrasound probe use while preserving the operator's ability to grip the ultrasound probe at different hand angles and stabilization of the hand on the patient's tissue. The interior of the shroud permits the operator's hand to take different grips and to accommodate different hand and probe sizes.

The shroud may be sized to fit around the hand grip to receive the hand of the healthcare professional therein.

It is thus a feature of at least one embodiment of the invention to optimize the trade-off between needle stick protection during simultaneous needle insertion and natural gripping of the ultrasound probe without interference.

The shroud may extend at least 100 mm from the probe axis at all angles about the probe axis.

It is thus a feature of at least one embodiment of the invention to permit the operator to adjust their grip, for example, to achieve different probe angles with respect to the patient's tissue thus permitting natural twisting and turning of the operator's hand grip.

The top end of the shroud may have a width of at least 150 mm. The bottom end of the shroud may have a width that is less than 50 mm.

It is thus a feature of the invention to provide an interior volume of the shroud resembling the natural tapering of the human operator's hand when gripping the probe. The shield may provide enough clearance to accommodate various and sometimes unconventional probe grips of the operator's hand.

The shroud may extend at least 180 degrees around the transducer.

It is thus a feature of at least one embodiment of the invention to provide protection at the front and sides of the ultrasound probe to protect the operator's hand during freehand insertion of the needle (without a needle guide) and insertion of the needle into the needle guide which may occur when the ultrasound probe is also being rotated for scanning.

The transducer may have four sides and the shroud may extend around at least three sides of the transducer.

It is thus a feature of at least one embodiment of the invention to ensure proper shielding with rectangular shaped ultrasound probes having four sides.

The connector may be a clamp configured to extend around a front end of the transducer housing.

It is thus a feature of at least one embodiment of the invention to minimize the interference of the connector with the operator's hand on a top end of the handgrip and permitting extension of the probe below the connector at a bottom end of the transducer without causing discomfort to the patient.

The shroud may have a bottom opening permitting extension of the front end of the transducer from a bottom of the shroud.

It is thus a feature of at least one embodiment of the invention to minimize the risk to needle stick but still allowing the bottom of the probe to be pressed into the patient's tissue during scanning.

The shroud may provide a cutout supporting extension of a corner of a front end of the transducer.

It is thus a feature of at least one embodiment of the invention to allow the shield to be used with ultrasound probes with built in or are attached to needle guides.

The shroud may provide a transparent window.

It is thus a feature of at least one embodiment of the invention to promote a line of sight of the needle with respect to the operator's hand, and the front end of the transducer with respect to the patient's tissue, which would normally be expected during training and ultrasound use without the shield.

The shroud may provide a frame supporting a replaceable film attachable to the frame. The replaceable film may be transparent allowing viewing therethrough. The replaceable film may be attachable to the frame by tabs receivable into corresponding holes.

It is thus a feature of at least one embodiment of the invention to permit a protective covering of the frame to be replaceable to prevent cross-contamination of the frame with the patient using a disposable film layer whereby the frame can be sterilized and reused. In an alternative embodiment of the present invention, the frame and film are manufactured as a unitary device that is disposable or sterilized and reused.

The shroud may be a curved cone with a wall curved around the transducer. The bottom end of the shroud may have curved corners.

It is thus a feature of at least one embodiment of the invention to provide an intuitive shroud resembling the shape of the operator's grip around the probe and minimizing discomfort to the patient when the probe is pressed into the tissue.

The invention may in addition or alternatively provide an ultrasound transducer having a housing supporting a hand grip extending along the axis and perpendicular to an array of piezoelectric elements of the transducer.

It is thus a feature of at least one embodiment of the invention to provide a hand shield that could be attached to and used with various commercial ultrasound transducers.

The invention may in addition or alternatively provide a needle guide attached to and extending outwardly from the transducer housing and providing a guide channel permitting a needle to extend into to the tissue of the patient below the transducer.

It is thus a feature of at least one embodiment of the invention to provide a hand shield that could be attached to and used with various commercial needle guides.

One embodiment of the present invention provides an ultrasound transducer comprising a housing supporting a hand grip extending along an axis perpendicular to an array of piezoelectric elements of the transducer; a needle guide attached to and extending outwardly from the transducer housing and providing a guide channel permitting a needle to extend into to the tissue of the patient below the transducer; and a hand shield providing a shroud supported by the transducer extending along the axis of the transducer and at least partially around the transducer to create a physical barrier between the hand grip and a needle insertable into the tissue of the patient below the transducer; and a connector supporting a connection of the shroud at a front end of the transducer and a space around the rear end of the transducer without contacting the transducer.

One embodiment of the present invention provides a disposable hand shield for an ultrasound transducer of a type having a shroud supported by the transducer and extending along the axis of the transducer to extend at least partially around the transducer to create a physical barrier between the hand grip and a needle insertable into the tissue of the patient below the transducer; and a connector supporting a connection of the shroud at a front end of the transducer and a space around the rear end of the transducer without contacting the transducer.

These particular objects and advantages may apply to only some embodiments falling within the claims and thus do not define the scope of the invention.

### BRIEF DESCRIPTION OF THE DRAWINGS

FIG. 1 is a perspective view of an ultrasound hand shield constructed according to the present invention and held by an operator's hand, the hand shield attached to an ultrasound probe with a needle holder and providing a frame attached to a front end of the ultrasound probe and supporting a transparent film blocking the operator's hand from a needle inserted through the needle holder into the patient's skin at an angle;

FIG. 2 is an exploded, perspective view of the ultrasound shield of FIG. 1 showing the replaceable film removed from the frame and an opening of the film and frame permitting the needle guide to extend to an outside of the ultrasound shield;

FIG. 3 is a perspective interior view of an alternative embodiment of the present invention, the ultrasound hand shield formed as a unitary shroud and a clamp of the ultrasound shield permitting the ultrasound shield to be attached to a front of the ultrasound probe;

FIG. 4 is a rear cross sectional view of the ultrasound hand shield of FIG. 3 showing a front of the ultrasound transducer extending below the shield to allow for various angles of insonation to be achieved; and

FIGS. 5A-5C are simplified, perspective views of various operator grips of an operator holding the ultrasound probe while the ultrasound shield is attached to the probe.

### DETAILED DESCRIPTION OF THE PREFERRED EMBODIMENT

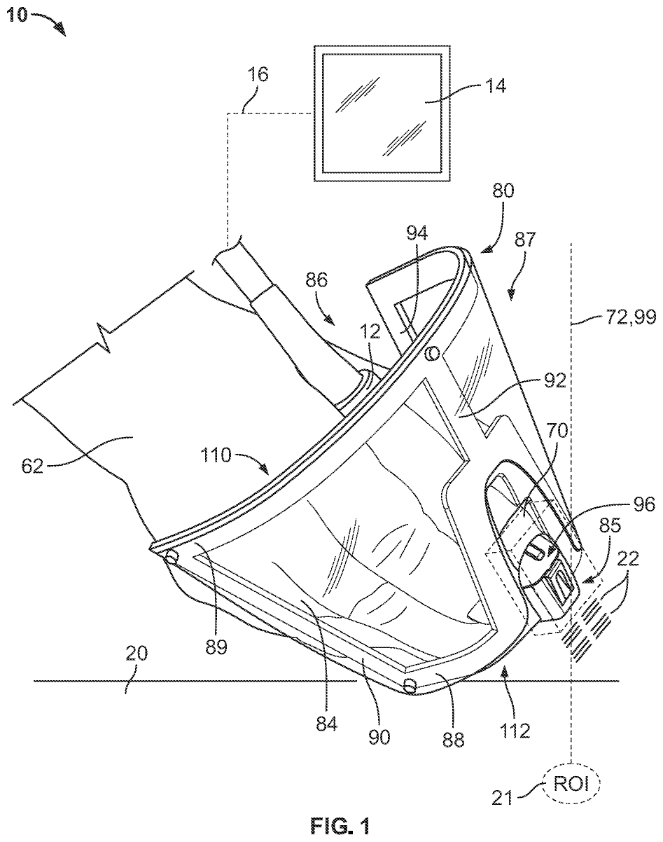

Referring now to FIG. 1, an ultrasound system 10 of the present invention may include an ultrasound transducer 12 for acquiring ultrasound echo data and communicating that ultrasound data with a display unit 14 for the display of information related to acquired ultrasound data. As shown, the transducer 12 and display unit 14 are separate so as to aid in the viewing of the display unit 14 during scanning of the transducer 12; however, it is contemplated that these two elements can be integrated into a single device. When separate, the transducer 12 may communicate with the display unit 14, for example, wirelessly by radio, ultrasound, or optical signals 16 or by means of a flexible electrical conductor. A keyboard may receive commands from an operator to provide information derived from ultrasonic measurements to be described.

During a typical medical procedure, the patient will lay down on an operating table, e.g., prone, supine, or on their side, and the operator will be standing or seated on the side of the operating table and patient to pass the transducer 12 over the patient tissue 20 and over a region of interest 21. It may be desired to position the display unit 14 in a position which minimizes the amount of operator head movement, for example, positioning the display unit 14 in a line of sight that minimizes side-to-side head movement of the operator but understandably may require some up-and-down head movement of the operator to view the display unit 14 and then the transducer 12 and patient tissue 20 below them during ultrasound scanning.

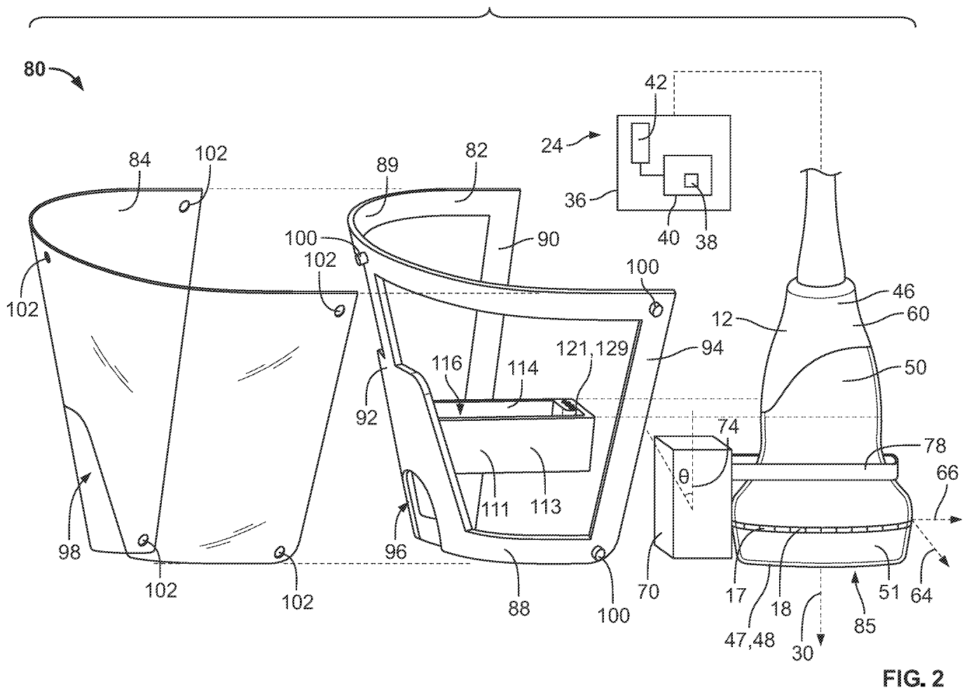

Referring also to FIG. 2, the ultrasound transducer 12 provides an array 17 of regularly spaced transducer elements 18 that each may convert a received electrical signal into an acoustic signal (or ultrasonic beams 22) delivered to the patient tissue 20, for example, through an aqueous gel or the like (not shown). The acoustic signals (or ultrasonic beams 22) are generally compression (longitudinal) ultrasonic signals that are in the same direction as the particle motion. The transducer elements 18 are also operated as sensors to receive ultrasonic acoustic energy to produce an electrical signal that may be measured, as further described below.

The ultrasound transducer 12 may provide for transducer elements 18 arrayed along a single dimension or a two-dimensional array. The transducer elements 18 are each capable of emitting ultrasonic acoustic waves into patient tissue 20 in contact with the underside of the array 17 and of receiving and detecting echo signals from those emitted ultrasonic acoustic waves for the generation of ultrasonic data. A signal processing system 24 generally controls the transducer elements 18 to provide separate diverging ultrasonic beams 22 that may be controlled in amplitude and phase for beamforming and other known techniques and are spaced along a longitudinal axis 66 of the transducer face 47. The signals necessary to produce the ultrasonic beams 22 and to measure the echo signals are generated by the signal processing system 24 using a programmed electronic processor 36 executing a stored program 38 contained in a computer memory 40. The resulting data from measurement of the propagation of the ultrasonic beams 22 may be stored as image or other data files 42.

The ultrasound transducer 12 may include a housing 46 supporting at a front end 85 of the transducer 12 an acoustic lens 48 forming a tissue contacting face 47 of the transducer 12 which may be a hard rubber that focuses the ultrasound waves into the contacting the patient tissue 20. Examples of hard rubber materials which may be used for the acoustic lens 48 include butadiene rubber, polybutadiene rubber, acrylic rubber and polyurethane, and the like.

Behind the acoustic lens 48 are several layers including a matching layer 51, the transducer elements 18, and a backing layer 50. The matching layer 51 and backing layer 50 sandwich the transducer elements 18 on a front side and a back side of the transducer elements 18, respectively.

The matching layer 51 help to transfer the ultrasound energy to the patient tissue 20 and reduces the impedance between the transducer elements 18 and the patient tissue 20. Examples of materials which may be used for the matching layer 51 include epoxy, polyurethane, polystyrene, parylene, composite materials, and the like.

The backing layer 50 suppresses vibrations of the transducer elements 18 and prevents them from ringing back into the transducer elements 18. Examples of materials which may be used for the backing layer 50 include graphite, porous graphite filled with resin, aluminum, and the like.

The housing 46 is acoustically insulated to prevent external soundwaves from interfering with the transducer 12. In some embodiments the housing 46 has a plastic outer shell with a metal shielding layer and acoustic insulator layer.

The housing 46 has a handle portion 60 receivable by a grip of the operator's hand 62 to extend generally along a central axis 30 behind and covering the backing layer 50. When held by the operator with the acoustic lens 48 extending downwardly to contact the patient tissue 20, the handle 60 may extend upwardly away from the array 17 of regularly spaced transducer elements 18 generally perpendicular to the array 17.

The handle 60 is manipulated with a light grip by the operator's hand 62 in a manner that, for example, orients the operator's hand 62 in a “pencil grip” configuration on the handle 60 whereby the operator's thumb and index finger squeeze the neck of the handle 60 across a narrower dimension of the handle 60 (the narrower dimension extending along a transverse axis 64 of the transducer face 47 perpendicular to the longitudinal axis 66 of the transducer face), and the underside of the handle 60 is supported by the operator's middle finger. The operator's pinky and ring finger may rest lightly on the patient tissue 20.

Ultrasound transducers of the type used with the present invention may be as described in U.S. Pat. Nos. 5,497,662 and 4,257,278, and as sold by GE Healthcare under “Probe-9L-D” found at <https://services.gehealthcare.com/gehcstorefront/p/5499510>, which define the scope of the prior art and are hereby incorporated by reference.

In some embodiments, a detachable needle guide 70 is attached at the front end 85 of the transducer 12 to improve needle 72 placement and visibility to the operator. The needle 72 may be aligned with the longitudinal axis 66 (not shown), or the transverse axis 64 of the transducer face of the transducer 12 (FIGS. 1 and 2). Therefore, a housing of the needle guide 70 may be attached to either the shorter or longer side of the transducer face 47 depending on the desired needle direction. In an exemplary embodiment, as seen in FIGS. 1 and 2, the needle guide 70 is a rectangular housing attached to and extending from the shorter side of the transducer 12 in order to align the needle 72 with the longitudinal axis 66 of the transducer face.

The needle guide 70 is commonly used in procedures, for example, for needle insertion in small body parts, tissue biopsies, fluid aspirations, kidney/liver biopsies and vascular access, to direct a needle 72 through a guide channel or sleeve 74 of the needle guide 70 along a predetermined direction and angle to a desired depth within the patient's tissue 20. The needle 72 is inserted through the guide sleeve 74 and then releasably clamped within the guide sleeve 74 of the needle guide 70 to be held in a fixed position within the guide sleeve 74 during the procedure at a predetermined angle with respect to the transducer 12. It is understood that the guide sleeve 74 may accommodate needles 72 with a range of different gauges (e.g., 14 to 20 gauge) and may alternatively support a catheter or guidewire desirably inserted into the patient tissue 20.

A long sterile plastic or latex shroud may be used to cover the transducer housing 46 before attaching the needle guide 70 to the transducer 12 to prevent contamination. The needle guide 70 may include a connector that permits secure attachment of the needle guide 70 to the transducer housing 46. In one embodiment as shown, the connector is a clamp ring 78 that extends around the transducer housing 46 and may be screw tightened or finger tightened to secure the clamp ring 78 around the transducer housing 46. In an alternative embodiment, the connector includes mating connector parts, e.g., male and female connector, of the needle guide and the transducer housing 46, respectively. In still another alternative embodiment, the needle guide 70 is permanently fixed to the transducer housing 46 for example being a part of the housing 46 or molded onto the housing 46.

A path 99 of the needle 72 with respect to the needle guide 70 may be visualized electronically on the display unit 14, as guidance lines seen on the ultrasound image, prior to needle insertion. The needle guide 70 and/or guide sleeve 74 may be adjusted to provide a predetermined, desired angle of insertion with respect to the transducer 12. For example, the guide sleeve 74 may be adjusted so that the tip or bevel of the needle 72 is angled (0), e.g., between 20 and 60 degrees, with respect to the acoustic lens 48 or tissue contacting face 47 of the transducer 12.

An insertion depth of the needle 72 can also be estimated or measured on the display unit 14, and thus the needle 72 length chosen or needle 72 position adjusted by sliding the needle 72 along the guide sleeve 74, to provide a desired depth of insertion into the tissue 20.

Needle guides of the type used with the present invention may be as described in U.S. Pat. Nos. 8,073,529, 8,449,531, and may be as sold by Civco under the trade name “Ultra-Pro II In-Plane Ultrasound Needle Guides-Multi-Angle” found at <https://www.civco.com/catalog/ultrasound-needle-guides/ultra-pro-2-ultrasound-needle-guides-multi-angle/>, which define the scope of the prior art and are hereby incorporated by reference.

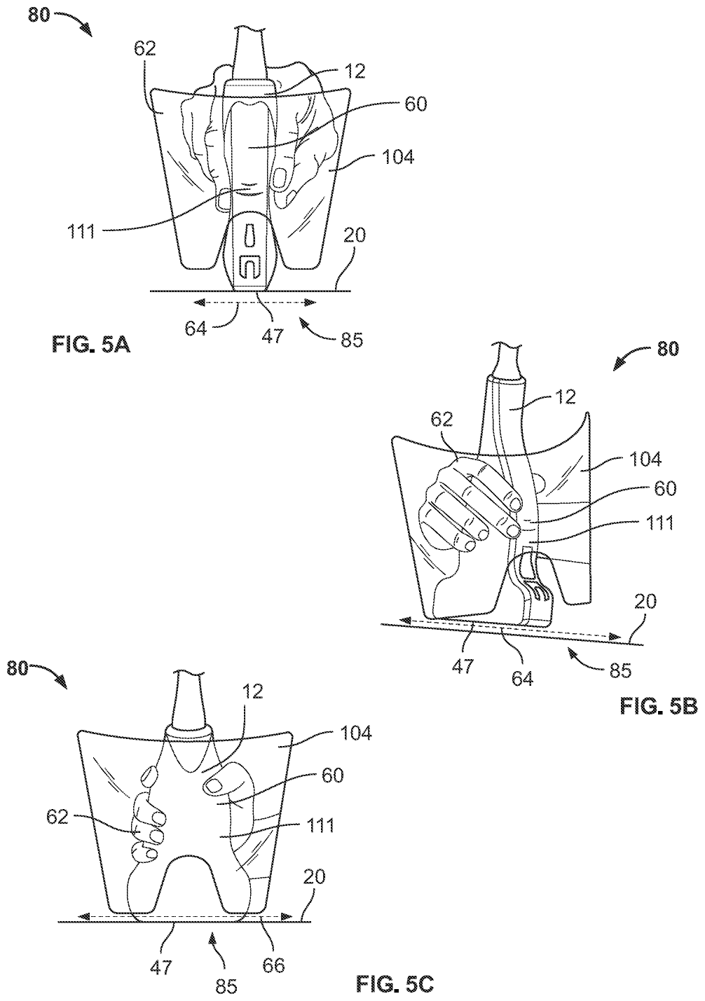

The needle 72, either freehandedly inserted or with the assistance of the needle guide as described above, may be inserted parallel to the transverse axis (across the ultrasound beam) or parallel to the longitudinal axis 66 (along the ultrasound beam). The needle 72 direction will depend on the applied anatomy, desired direction of needle insertion, ease of visualization of structures with ultrasound, and operator skill and experience. In one embodiment of the present invention, as shown in FIG. 1, the needle 72 is inserted along the transducer array elements 18, parallel to the longitudinal axis 66, such that the needle 72 is parallel to the ultrasonic beam 22. In another embodiment of the present invention, as shown in FIG. 5C, the needle 72 is inserted perpendicular to the transducer array elements 18, parallel to the transverse axis 64, such that the needle 72 intersects the ultrasonic beam 22 at a certain depth.

Referring to FIGS. 1 and 2, the present invention provides a hand shield assembly 80 which in some embodiments provide a frame 82 supporting a transparent film 84 that may be removably attached to the frame 82. A connector may be used to attach the frame 82 to the transducer 12 at the front end 85 of the transducer 12 near the acoustic lens 48 of the transducer 12 to allow for normal handling of the handle 60 by the operator without obstruction.

The frame 82 of the hand shield assembly 80 may take a curved conical shape curved around the transducer housing 46 which in turn supports the flexible, transparent film 84 curved around the frame 82 and forms a shield or curved wall positioned between (a) the handle 60 and the operator's hand 62 on an interior 86 of the curved transparent film 84 (i.e., concave side) and (b) the needle 72 and the path 99 of the needle 72 as it is being inserted into the patient's tissue 20 on an exterior 87 of the curved transparent film 84 (i.e., convex side). In this respect, the hand shield assembly 80 provides a protective barrier between the operator's hand 62 and the needle 72 minimizing the risk to needle sticks.

The frame 82 may include an upper curved band 89 attached to a lower curved band 88 by transverse column members 90, 92, 94 supporting connection of the upper curved band 89 and the lower curved band 88. The outer left and right column members 90 and 94 define the outer left and right edges of the frame 82, respectively, and the central column member 92 supports a center of the frame 82. The upper curved band 89 and the lower curved band 88 are semi-circular, semi-oval, or semi-elliptical bands that curve at least 180 degrees to shield at least three sides (front facing side and lateral sides) of the rectangular transducer housing 46. The upper curved band 89 has a radius of curvature that is greater than the lower curved band 88 to support a wider portion of the operator's hand 62 when gripping the transducer 12 which narrows or tapers toward the front end 85 of the transducer 12.

A center of the lower curved band 88 located between the outer left and right column members 90 and 94 may include a cutout 96 framed by the lower curved band 88 curving upward into an arch shape which permits the transducer 12 and the attached needle guide 70 to protrude outwardly from the interior of the frame 82. The cutout 96 may be a rounded arch permitting a side or a bottom corner of the transducer housing 46 to extend out thought the cutout 96. The height and width of the cutout 96 may be between 30 and 50 mm in height and between 30 and 50 mm wide to accommodate the size and shape of the transducer housing 46 (and part of the needle guide 70). In this respect, a portion of the transducer housing 46, and the needle guide 70, if attached, may extend to the exterior 87 of the frame 82 and the hand shield assembly 80 may be used in the presence of a needle guide 70. It is understood that the cutout 96 may take different shapes or sizes such as a rectangular or circular shape which resemble and accommodate the shape or the size of the extending transducer housing 46 and/or needle guide 70.

Referring still to FIGS. 1 and 2, but best seen in FIG. 2, the transparent film 84 may be a clear film of, e.g., polyester film, polycarbonate, polyethylene terephthalate, and the like, which provides high transparency, is bendable/flexible around the frame 82, is easy to clean, and can be die cut to be easily manufactured and attached to the frame 82. The transparent film 84 may be curved to bend around the outside of the frame 82 and attached to the frame 82 to provide a physical barrier.

The transparent film 84 generally conforms to the shape of the frame 82, aligning to the shape of a perimeter of the frame 82 and providing the same indentations and cutouts. The transparent film 84 may take a curved conical shape resembling the frame 82. The transparent film 84 provides a corresponding cutout 98 which aligns with the cutout 96 of the frame 82 and permitting the transducer housing 46 and the needle guide 70 to extend outwardly to the exterior 87 of the frame 82 past the transparent film 84.

The transparent film 84 may be attached to the frame 82 by various connection methods which allow the transparent film 84 to be easily removed and replaced. In one embodiment, the outer surface of the frame 82 may include outwardly extending notches 100 which may be inserted into corresponding holes 102 of the transparent film 84 allowing the transparent film 84 to be attached to frame 82 by punching or pushing the notches 100 through the perforated holes 102. The notches 100 of the frame 82 may be positioned at the corners of the frame 82 and at the center of the frame 82 to ensure that the transparent film 84 is securely attached along the outer perimeter of the frame 82 and inside the concave channel 97.

In an alternative embodiment, the outer surface of the frame 82 may receive an adhesive, such as a glue, tape or Velcro, which may allow the transparent film 84 to stick to the outside frame 82. It is understood that other transparent film 84 attachment methods may be used and are contemplated as being within the scope of the present invention.

The transparent film 84 may be removable from the frame 82 and disposable. The frame 82 may then be sterilized to be reused with a new transparent film 84.

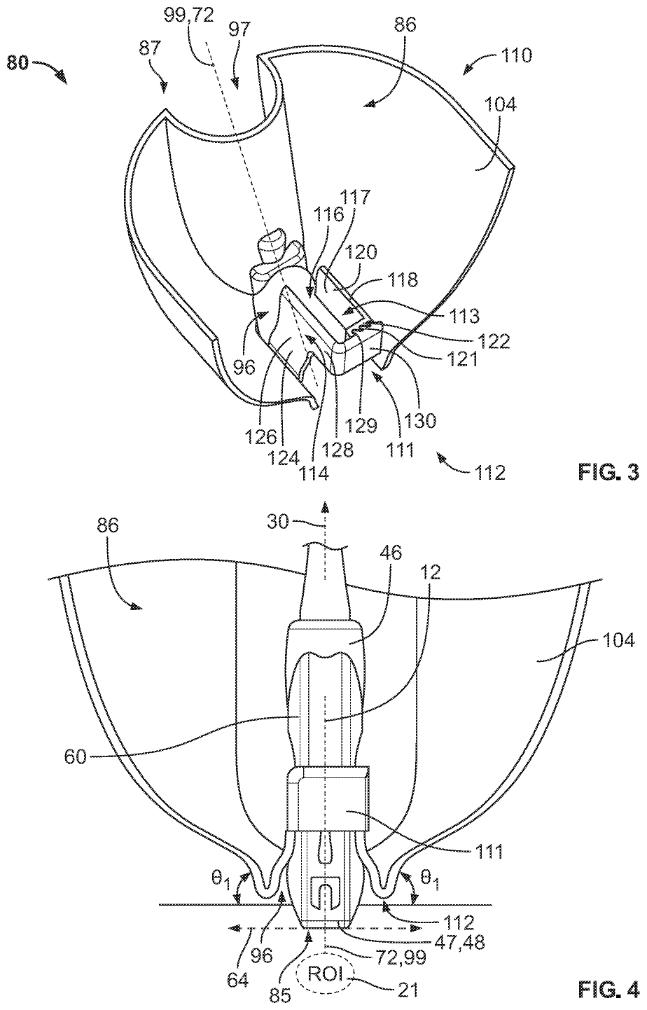

Referring now to FIGS. 3 and 4, it is understood that in an alternative embodiment, the frame 82 and the transparent film 84 may be integrally formed as a unitary piece such that the entire hand shield assembly 80 is disposable or sterilized to be reused. In this respect, the hand shield assembly 80 may be manufactured of a molded shroud 104 providing full transparency or at least some partial transparency or transparent windows within the shroud 104 of the hand shield assembly 80.

The shroud 104 may take an outer shape that is similar to the perimeter of the frame 82 as described above but may be a solid material that provides a shield or curved wall between the handle 60 and the operator's hand 62 on an interior 86 of the shroud 104 (i.e., concave side) and the needle 72 and the path 99 of the needle 72 as it is being inserted into the patient's tissue on an exterior 87 of the shroud 104 (i.e., convex side). The wall of the shroud 104 generally conforms to the shape of the frame 82 and/or transparent film 84 to provide a curved conical shape around the transducer 12.

The shroud 104 provides a similar cutout 96 as described above with respect to the frame 82 which allow the transducer housing 46 and needle guide 70 to extend outwardly from the interior 86 of the shroud 104 and exposing the needle guide 70.

Further, in some embodiments, a concave channel 97 may also be formed within a front surface of the shroud 104, the concave channel 97 extending from the top to the bottom of the shroud 104 and centered between the left and right edges of the shroud 104. The concave channel 97 may be formed by a semi-cylindrical, inward indentation formed directly within the front surface of the shroud material. In this respect, the needle 72 may extend on an exterior 87 of the shroud 104 and along the pathway 99 through the concave channel 97 and into the needle guide 70 without interference from the frame 82. Other similar indentations may be formed within the shroud material in order to accommodate the transducer 12, needle 72, or needle guide on the exterior 87 of the shroud 104.

It is understood that if the concave channel 97 was applied to the frame 82 and transparent film 84 as shown in FIGS. 1 and 2, the concave channel 97 may be formed by an inward bend within the center upper curved band 89 and the lower curved band 88. The central column member 92 at the center of the frame 82 therefore may also be positioned inwardly to permit the clear pathway 99 of the needle 72 into the needle guide 70. The transparent film 84 may then also bend inwardly into the concave channel 97 of the frame 82 to align with the inward bend of the frame 82 and permitting the needle 72 to be guided through the concave channel 97 and into the needle guide 70 on the exterior of the transparent film 84.

Referring now to FIGS. 1 through 4, the curved corners of the frame 82, formed at the outer edges of the upper curved band 89 and the lower curved band 88 or by the corners of the shroud 104 are rounded to eliminate any sharp corners from contacting the patient tissue 20 or the operator's hand 62 during ultrasound scanning and causing discomfort.

The hand shield assembly 80, formed either by the frame 82 and transparent film 84 as seen in FIGS. 1 and 2, or the unitary shroud 104 as seen in FIGS. 3 and 4, may have a shape providing an upper end 110 that is wider than the lower end 112 and thus tapering downward to the front end 85 of the transducer 12.

The upper end 110 of the frame 82 (and transparent film 84) or shroud 104 has an upper width, e.g., extending between the outer edges of the upper curved band 89, that is between 100 mm and 200 mm wide and between 150 mm and 200 mm wide and between 150 mm and 180 mm wide and approximately 165 mm wide. The lower end 112 of the frame 82 (and transparent film 84) or shroud 104 has a lower width, e.g., extending between the outer edges of the lower curved band 88, that is between 40 mm and 100 mm wide and between 40 mm and 80 mm wide and between 40 mm and 60 mm wide and approximately 50 mm wide. The lower end 112 of the frame 82 (and transparent film 84) or shroud 104 is narrower than the upper end 110 of the frame 82 (and transparent film 84) or shroud 104 to allow for better tilt of the transducer 12 and, in particular, to provide a desired angle of insonation (01) of the acoustic lens 48 with respect to the patient tissue 20 for proper imaging.

A depth of the frame 82 (and transparent film 84) or shroud 104, e.g., correlating with the radius of curvature of the upper curved band 89 and the lower curved band 88, is between 50 mm and 150 mm and between 80 mm and 130 mm and 90 mm and 120 mm and approximately 115 mm.

A height of the frame 82 (and transparent film 84) or shroud 104, e.g., extending between the upper curved band 89 and the lower curved band 88 is between 50 mm and 150 mm and between 90 mm and 140 mm and 100 mm and 130 mm and approximately 100 mm. The height of the frame 82 or shroud 104 may allow for the entire hand of the operator or most of the operator's hand 62 to be shielded.

The width, depth, and height of the hand shield assembly 80 may approximate average proportions of the operator's hand 62 when gripping the handle 60 of the transducer 12 but providing additional space to accommodate for variations in hand size and grip. For example, the width, depth, and height of the hand shield assembly 80 may approximate the average width, depth, and height of the operator's hand 62 when gripping the handle 60 of the transducer 12. In one embodiment and depending on the size of the transducer housing 46 and the operator's grip, an average width and height of the operator's hand 62 when gripping the handle is approximately 100 mm to 150 mm in width, 70 mm to 100 mm in depth, and 60 mm to 90 mm in height. Therefore, the width, depth, and height of the hand shield assembly 80 may be at least as large as the dimensions of the average width and height of the operator's hand 62 when gripping the handle 60. Each of the dimensions may be at least 5% and at least 10% and at least 15% and at least 20% larger than the dimensions of the average width and height of the operator's hand 62 when gripping the handle 60.

The hand shield assembly 80 may extend from the central axis 30 of the transducer 12 at least 100 mm and at least 110 mm and at least 120 mm and at least 130 mm and at least 140 mm and at least 150 mm.

The volume formed within the interior 86 of the hand shield assembly 80 may be at least 5% and at least 10% and at least 15% and at least 20% larger than the volume of the operator's hand 62 when gripping the handle 60 to allow the interior 86 to comfortably accommodate the operator's hand. Therefore, it is contemplated that the interior 86 of the hand shield assembly 80 provides a volume sufficient to allow the operator's hand 62 to be supported therein and the volume is at least as large as the volume of the operator's hand 62 when gripping the handle 60.

Referring specifically to FIG. 3, the hand shield assembly 80 is attached at the front end 85 of the transducer 12 by a connector which may be a clamp 111 receiving the front end 85 of the transducer 12. The clamp 111 is attached at the bottom of the frame 82, for example, to the lower curved band 88, or the lower edge of the shroud 104. The clamp 111 provides a pair of inwardly extending arms 113, 114 biased inwardly toward a center of the interior 86 of the frame 82.

In one embodiment, the clamp 111 provides a first clip arm 113 and an opposed, mating second clip arm 114. The first clip arm 113 is attached at a left side of the frame 82 or shroud 104, and the second clip arm 114 attached at a right side of the frame 82 or shroud 104 and which when placed together form a rectangular shaped grip opening 116.

The first clip arm 113 may provide a vertically extending member 117 further connected to a horizontally extending L-shaped member 118. The horizontally extending L-shaped member 118 has a first member 120 and a second member 122 forming the L-shape, the first arm connected to the vertically extending member 117 at a top end and extending in a rearward direction to the perpendicular second member 122 which provides interlocking teeth 121 on an outer surface of a second member 122. In some embodiments, for example as shown in FIG. 1, the vertically extending member 117 is eliminated.

The second clip arm 114 may provide a vertically extending member 124 connected to a horizontally extending L-shaped member 126. The horizontally extending L-shaped member 126 has a first member 128 and a second member 130 forming the L-shape, the first member 128 connected to the vertically extending member 124 at a top end and extending in a rearward direction to the perpendicular second member 130 which provides interlocking teeth 129 on an inner surface of the second member 130. In some embodiments, for example as shown in FIG. 1, the vertically extending member 124 is eliminated.

The L-shaped member 118 of the first clip arm 113 and the L-shaped member 126 of the second clip arm 114 form a three sided rectangle surrounding the rectangular shaped grip opening 116, with the first members 120 and 128 of the first clip arm 113 and the second clip arm 114, respectively, forming opposed sides of the rectangle and the second members 122 and 130 of the first clip arm 113 and the second clip arm 114, respectively, forming a third side of the rectangle.

The second members 122 and 130 of the first clip arm 113 and the second clip arm 114, respectively, overlap, with the second member 130 of the second clip arm 114 on the outside and the second member 122 of the first clip arm 113 on the inside, thus permitting the outer interlocking teeth 121 of the second member 122 to engage with the inner interlocking teeth 129 of the second member 130 to interlock. The interlocking of the teeth 121 and 129 enclose the rectangular shaped grip opening 116 and secure the clamp 111 around the transducer 12.

The enclosed rectangular shaped grip opening 116 is sized to receive the front end 85 of the transducer 12 so that the clamp 111 can securely grip the transducer housing 46. The multiple teeth along the transverse axis 64 of the interlocking teeth 121, 129 permit a width of the rectangular shaped grip opening 116 extending along the transverse axis 64 to be varied in order to accommodate various dimensions of the transducer face 47. For example, an engaging of interlocking teeth 121, 129 at more distal ends of the second members 122 and 130 will accommodate a wider transducer face 47 and likewise, an engaging of interlocking teeth 121, 129 at more proximal ends of the second members 122 and 130 will support a narrower transducer face 47.

It is understood that the first clip arm 113 and the second clip arm 114 are tensioned outwardly by the transducer housing 46 positioned therein to keep the interlocking teeth 121, 129 engaged, but may be “unhooked” by applying an inward and then outward force to release the interlocking teeth 121, 129 from engagement. The interlocking teeth 121, 129 may take an interlocking wave-shaped profile as shown but understandably may take other profile shapes allowing for similar variable width, interlocking engagement.

Referring specifically to FIG. 4, in operation, the first clip arm 113 and the second clip arm 114 may extend around the transducer housing 46 slightly rearwardly from the acoustic lens 48 so that the acoustic lens 48 extends below the lower end 112 of the hand shield assembly 80 thus permitting the acoustic lens 48 and the front end 85 of the transducer 12 to be pressed, tilted, or rocked in the patient tissue 20 without interference from the hand shield assembly 80. The acoustic lens 48 may extend at least 5 mm and at least 10 mm and at least 15 mm below the lower end 112 of the hand shield assembly 80.

It is understood that other types of connectors may be used to attach the front end 85 of the transducer 12 to the hand shield assembly 80. For example, different types of clamps, clips, mating connectors, and the like may be used. The connector desirably provides an unobstructed space or gap around the entire handle 60 of the transducer 12 which the operator can grip freely without obstruction from the connector. The connector does not contact the transducer 12 at the rear end opposite the front end 85 to avoid any interference. The connector further allows the front end 85 of the transducer 12 to extend downwardly below the connector and past the lower end 112 of the hand shield assembly 80. The connector further accommodates various sizes and widths of the transducer 12 by providing a flexible grip opening 116 to grip the transducer 12.

Referring now to FIGS. 5A to 5C, the various hand grips of the operator's hand 62 on the transducer 12 may be accommodated by the shape and size of the hand shield assembly 80. Exemplary operator hand grips are shown in FIGS. 5A to 5C.

Referring to FIG. 5A, as shown, the “palmar grip” positions the ultrasound transducer 12 between the operator's index and middle finger. The operator's fingers pinch or grip the transducer 12 across the transverse axis 64 of the transducer 12.

Referring to FIG. 5B, as shown, the “longitudinal grip” positions the ultrasound transducer 12 between the operator's index finger and thumb. The operator's fingers pinch or grip the transducer 12 across the transverse axis 64 of the transducer 12. The operator's ulnar aspect of the hand may rest upon the patient tissue 20.

Referring to FIG. 5C, as shown, the “transversal grip” positions the ultrasound transducer 12 between the operator's index finger (and optionally, the middle finger) and the operator's thumb. While similar to the longitudinal grip of FIG. 5B, this transversal grip positions the pinching or squeezing fingers across the longitudinal axis 66 of the transducer face 47 rather than the transverse axis 64. The operator's ulnar aspect of the hand may rest upon the patient's tissue 20.

When the transducer 12 is handled using the transversal grip, the hand shield assembly 80 may also be attached to curve around the longer dimension of the face of the transducer 12. In this respect, it is understood that the dimensions of the shroud 104 may correspond with and accommodate the dimensions of the longer face of the transducer 12, and optionally, the extension of the needle guide 70 from the longer side of the transducer 12 by providing the cutout 96 as described above.

In operation, the transducer 12 is held by the hand shield assembly 80 by locking the front end 85 of the transducer 12 into the clamp 111 of the hand shield assembly 80. The side or bottom corner of the front end 85 of the transducer housing 46 extends through the cutout 96 of the hand shield assembly 80, from the interior 86 to the exterior 87 of the hand shield assembly and permit the attached needle guide 70 of the transducer 12 (not shown in FIGS. 5A-5C) to extend on the exterior 87 of the hand shield assembly 80.

The operator will grip the handle 60 of the transducer 12, e.g., as shown in FIGS. 5A to 5C, on the interior 86 of the hand shield assembly 80 without interference from the clamp 111 or the frame 82 (and transparent film 84) or shroud 104. The operator will slide the transducer 12 over the patient tissue 20, for example, pressing, tilting and rocking the transducer 12 and transducer face 47 into the patient tissue 20. When the region of interest 21 inside the patient and beneath the transducer 12 is located on the display unit 14, the operator may insert the needle 72 through the needle guide 70 attached to the transducer 12 at a desired angle of insonation and depth into the patient tissue 20. The path 99 of the needle 72 may pass through the concave channel 97 of the hand shield assembly 80. Therefore, the hand shield assembly 80 will create a blocking wall or barrier between the operator's hand 62 and the needle 72 minimizing the risk of needle sticks.

It is understood that the present invention contemplates use of the hand shield assembly 80 with various commercially available ultrasound transducers 12 and needle guides 70. Therefore, the hand shield assembly may be a “one size fits all” for many or most ultrasound transducer 12 and/or needle guides 70.

The hand shield assembly 80 may be sold with the ultrasound transducer 12 and/or the needle guides 70 as part of a kit which may be sized to a particular size and shape of the ultrasound transducer 12 and/or needle guide 70.

Certain terminology is used herein for purposes of reference only, and thus is not intended to be limiting. For example, terms such as “upper”, “lower”, “above”, and “below” refer to directions in the drawings to which reference is made. Terms such as “front”, “back”, “rear”, “bottom” and “side”, describe the orientation of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first”, “second” and other such numerical terms referring to structures do not imply a sequence or order unless clearly indicated by the context.

When introducing elements or features of the present disclosure and the exemplary embodiments, the articles “a”, “an”, “the” and “said” are intended to mean that there are one or more of such elements or features. The terms “comprising”, “including” and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

References to “a controller” and “a processor” can be understood to include one or more microprocessors that can communicate in a stand-alone and/or a distributed environment(s), and can thus be configured to communicate via wired or wireless communications with other processors, where such one or more processor can be configured to operate on one or more processor-controlled devices that can be similar or different devices. Furthermore, references to memory, unless otherwise specified, can include one or more processor-readable and accessible memory elements and/or components that can be internal to the processor-controlled device, external to the processor-controlled device, and can be accessed via a wired or wireless network.

It is specifically intended that the present invention not be limited to the embodiments and illustrations contained herein and the claims should be understood to include modified forms of those embodiments including portions of the embodiments and combinations of elements of different embodiments as come within the scope of the following claims. All of the publications described herein, including patents and non-patent publications are hereby incorporated herein by reference in their entireties.

To aid the Patent Office and any readers of any patent issued on this application in interpreting the claims appended hereto, applicants wish to note that they do not intend any of the appended claims or claim elements to invoke 35 U.S.C. 112(f) unless the words “means for” or “step for” are explicitly used in the particular claim.

What we claim is:

1. An ultrasound transducer hand shield for protecting a hand of a healthcare professional gripping a transducer in a scanning position with a front end of the transducer adjacent to tissue of a patient supporting an array of piezoelectric elements, and a rear end of the transducer supporting a hand grip extending along an axis of the transducer perpendicular to the array of piezoelectric elements, the hand shield comprising:
a shroud removably supported by the transducer to extend along the axis of the transducer and at least partially around the transducer to create a physical barrier between the hand grip and a needle insertable into the tissue of the patient below the transducer; and
a connector supporting a connection of the shroud at a front end of the transducer
wherein the shroud extends around but does not contact the rear end of the transducer.

2. The hand shield of claim 1 wherein the shroud is sized to fit around the hand grip to receive the hand of the healthcare professional therein.

3. The hand shield of claim 1 wherein the shroud extends at least 100 mm from the axis at all angles about the axis.

4. The hand shield of claim 1 wherein a top end of the shroud has a width of at least 150 mm.

5. The hand shield of claim 1 wherein a bottom end of the shroud has a width that is less than 50 mm.

6. The hand shield of claim 1 wherein the shroud extends at least 180 degrees around the transducer.

7. The hand shield of claim 1 wherein the transducer has four sides and the shroud extends around at least three sides of the transducer.

8. The hand shield of claim 1 wherein the connector is a clamp configured to extend around a front end of the transducer housing.

9. The hand shield of claim 1 wherein the shroud has a bottom opening permitting extension of the front end of the transducer from a bottom of the shroud.

10. The hand shield of claim 1 wherein the shroud provides a cutout supporting extension of a corner of a front end of the transducer

11. The hand shield of claim 1 wherein the shroud provides a transparent window.

12. The hand shield of claim 1 wherein the shroud provides a frame supporting a replaceable film attachable to the frame.

13. The hand shield of claim 12 wherein the replaceable film is transparent allowing viewing therethrough.

14. The hand shield of claim 12 wherein the replaceable film is attachable to the frame by tabs receivable into corresponding holes.

15. The hand shield of claim 1 wherein the shroud is a curved cone with a wall curved around the transducer.

16. The hand shield of claim 15 wherein a bottom end of the shroud has curved corners.

17. The hand shield of claim 1 further comprising an ultrasound transducer having a housing supporting a hand grip extending along the axis and perpendicular to an array of piezoelectric elements of the transducer.

18. The hand shield of claim 17 further comprising a needle guide attached to and extending outwardly from the transducer housing, and providing a guide channel permitting a needle to extend into to the tissue of the patient below the transducer.

19. An ultrasound transducer for use on tissue of a patient comprising:
a housing supporting a hand grip extending along an axis perpendicular to an array of piezoelectric elements of the transducer;
a needle guide attached to and extending outwardly from the transducer housing and providing a guide channel permitting a needle to extend into to the tissue of the patient below the transducer; and
a hand shield providing
a shroud supported by the transducer extending along the axis of the transducer and perpendicular to the axis of the transducer configured to extend around the transducer and to create a physical barrier between the hand grip and a needle insertable into the tissue of the patient below the transducer; and
a connector supporting a connection of the shroud at a front end of the transducer and a space around a rear end of the transducer without contacting the transducer.

20. A disposable hand shield for an ultrasound transducer used on tissue of a patient of a type comprising:
a shroud supported by the transducer extending along a central axis of the transducer and to extend at least partially around the transducer to create a physical barrier between the hand grip and a needle insertable into the tissue of the patient below the transducer; and
a connector supporting a connection of the shroud at a front end of the transducer and a space around a rear end of the transducer without contacting the transducer.
